(19) United States
(12) Patent Application Publication
(10) Pub. No.: US 2012/0046536 A1
(43) Pub. Date: Feb. 23, 2012

(54) Surgical Instrument Navigation Systems and Methods

(75) Inventors: Andrew Cheung, Knoxville, TN (US); Joshua Campbell, Knoxville, TN (US)

(73) Assignee: Manhattan Technologies, LLC, Oak Ridge, TN (US)

(21) Appl. No.: 12/860,635

(22) Filed: Aug. 20, 2010

(51) Int. Cl. A61B 5/05

(52) U.S. Cl. 600/407

(57) ABSTRACT

A navigation system to track positions of surgical components during surgery of a patient. The navigation system includes a power source to emit a tracking signal during surgery of the patient, a first sensor mounted to a movable region of the patient to respond to the emitted tracking signal, and a control unit to track a position of the movable region relative to a fixed region of the patient as the movable region moves with respect to the fixed region, based on the response of the first sensor. The system can calibrated and register a movable reference point of the patient relative to a fixed reference point, and can maintain that reference point when the movable reference point moves in space during a surgical process.

### BACKGROUND OF THE INVENTION

1. Field of Invention

The present inventive concept relates generally to surgical instruments and, more particularly, to systems and methods to assist a surgeon in navigating anatomical regions of a patient to properly position surgical instruments during surgery.

2. Description of the Related Art

The controlled positioning of surgical instruments is of significant importance in many surgical procedures, and various methods and navigation systems have been developed to navigate a surgical instrument relative to a patient during surgery. Intra-operative navigation systems are comparable to global positioning satellite (GPS) systems commonly used in automobiles and are composed of three primary components: a localizer, which is analogous to a satellite in space; an instrument or surgical probe, which represents the track waves emitted by the GPS unit in the vehicle; and CT scan data set that is analogous to a road map of the anatomical structure of the patient. These image navigation techniques generally allow positioning of a surgical instrument within a margin of error of about 1 to 2 mm.

Computer assisted image guidance techniques typically involve acquiring preoperative images of the relevant anatomical structures and generating a data base which represents a three dimensional model of the anatomical structures. The position of the instrument relative to the patient is determined by the computer using at least three fixed reference elements that span the coordinate system of the object in question. The process of correlating the anatomic references to the digitalized data set constitutes the registration process. The relevant surgical instruments typically have a known and fixed geometry which is also defined preoperatively. During the surgical procedure, the position of the instrument being used is registered with the anatomical coordinate system and a graphical display showing the relative positions of the tool and anatomical structure may be computed and displayed to assist the surgeon in properly positioning and manipulating the surgical instrument with respect to the relevant anatomical structure.

One of the disadvantages of known systems is the need to maintain proper positioning of surgical instruments relative to movable anatomic references when those references are moved during surgery, and to enable surgeons to properly position surgical instruments in real time when anatomical reference points are moved during surgery.

### BRIEF SUMMARY OF THE INVENTION

The present general inventive concept provides systems and methods to digitally register and track movable regions of a patient, enabling a surgeon to accurately position and navigate surgical instruments with respect to movable reference points even when the movable reference points are moved in space during the surgical procedure.

Additional features and embodiments of the present general inventive concept will be set forth in part in the description which follows and, in part, will be obvious from the description, or may be learned by practice of the general inventive concept.

Example embodiments of the present general inventive concept can be achieved by providing a navigation system to track positions of surgical instruments during surgery of a patient, including a power source to emit a detectable signal during surgery of a patient, a first sensor mounted to a movable region of the patient to respond to the emitted signal, and a control unit to track a position of the movable region relative to a fixed region of the patient as the movable region moves with respect to the fixed region, based on the response of the first sensor.

The navigation system can include a second sensor mounted to a surgical instrument to respond to the emitted signal such that the control unit tracks a position of the surgical instrument relative to the movable region as the surgical instrument and movable region move with respect to the fixed region, based on the responses of the first and second sensors.

Example embodiments of the present general inventive concept can also be achieved by providing a navigation system to track positions of surgical instruments during surgery of a patient, including a detection unit to detect an LED signal, a first sensor mounted to a movable region of the patient to emit a first LED signal to be detected by the detection unit, and a control unit to track a position of the movable region relative to a fixed region of the patient as the movable region moves with respect to the fixed region, based on the detected first LED signal.

Example embodiments of the present general inventive concept can also be achieved by providing a method of tracking positions of surgical instruments during a surgical process of a patient, including emitting tracking signals to a targeted region of the surgical process, coupling a first sensor to a movable region of the patient such that the first sensor responds to the emitted tracking signals, and tracking a position of the movable region relative to a fixed region of the patient as the movable region moves with respect to the fixed region, based on the response of the first sensor.

### BRIEF DESCRIPTION OF THE SEVERAL VIEWS OF THE DRAWINGS

The above-mentioned features of the present general inventive concept will become more clearly understood from the following detailed description read together with the drawings in which:

FIG. 1 is a perspective view of a system environment in which the features of the present general inventive concept may be implemented;

FIG. 2A is a perspective view of a guide member including sensor members in accordance with an example embodiment of the present general inventive concept;

FIG. 2B is a perspective view of guide member including sensor members in accordance with another example embodiment of the present general inventive concept;

FIG. 3 is a perspective view of a surgical instrument including sensor members in accordance with an example embodiment of the present general inventive concept; and

FIG. 4 is a diagram illustrating a power source emitter and detection unit communicating with sensor units in accordance with example embodiments of the present general inventive concept.

### DETAILED DESCRIPTION OF THE INVENTION

Reference will now be made to various embodiments of the present general inventive concept, examples of which are illustrated in the accompanying drawings, wherein like reference numerals refer to like elements throughout. The following description of the various embodiments is merely exemplary in nature and is in no way intended to limit the present general inventive concept, its application, or uses. The example embodiments are merely described below in order to explain the present general inventive concept by referring to the figures.

The present general inventive concept provides systems and methods of tracking a location of a movable reference point relative to a fixed reference point as the movable reference point moves in space with respect to the fixed reference point during a surgical procedure.

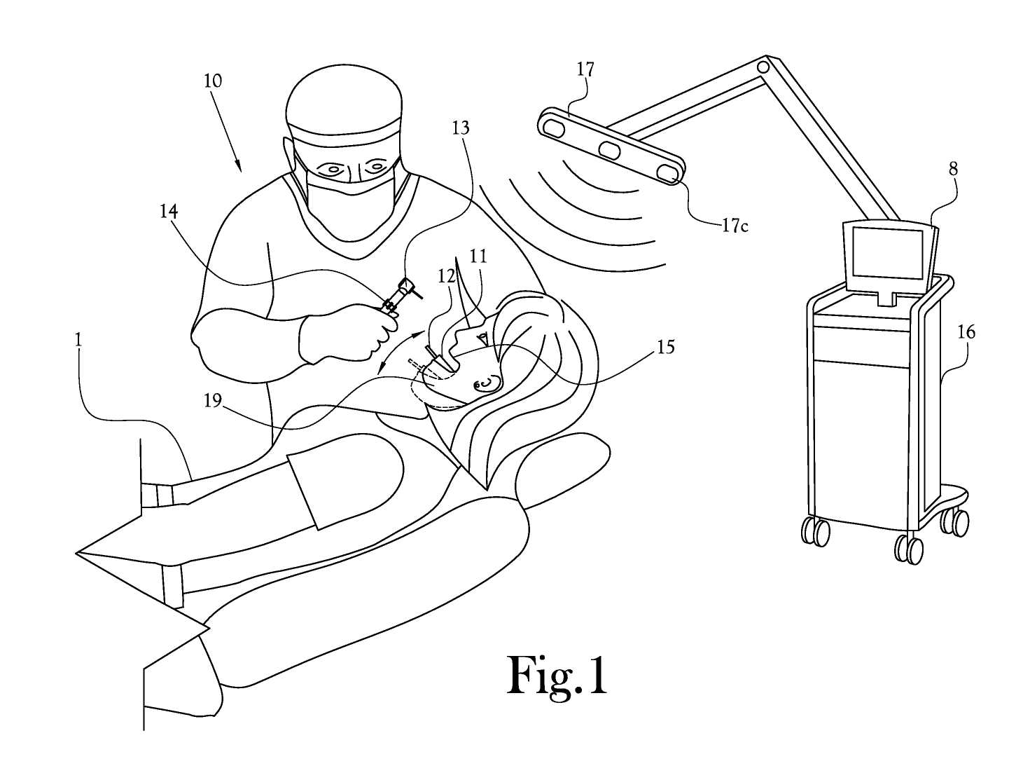

FIG. 1 is a perspective view illustrating an exemplary system environment in which the features of the present general inventive concept may be implemented. The system environment of FIG. 1 includes a navigation system generally indicated by reference number 10 to navigate surgical instruments with respect to targeted anatomical structures of a patient 1. The simplified diagram of FIG. 1 illustrates a drilling instrument 13 for use in an oral surgery procedure and a patient 1. In FIG. 1, the patient is prepared for oral surgery toward a targeted region of the patient's mandible 19. As illustrated in FIG. 1, the mandible 19 is a movable anatomical structure as generally indicated by the phantom lines and direction arrow in FIG. 1. Since the mandible 19 is movable with respect to a fixed reference point such as the patient's skull or maxilla 15, the mandible 19 is referred to as a movable region or movable reference point. However, the present general inventive concept is not limited to any particular anatomical structure or type of movable reference point, nor is it limited to oral surgery procedures. Those skilled in the art will appreciate that many other anatomical structures could be used as a movable reference depending on the location and scope of the targeted surgical region, such as head, legs, arms, feet, hands, etc. Accordingly, the present general inventive concepts can be used to navigate any type of surgical or medical/dental instrument, for example, endoscopic systems, suction devices, screw devices, guides, wires, syringes, needles, drug delivery systems, biopsy systems, arthroscopic systems, etc. Furthermore, the surgical instruments of the present general inventive concept may be used to navigate any targeted region or anatomical structure of the patient's body during any medical or dental procedure, internally or externally, in addition to surgery on the mandible region as illustrated in FIG. 1. It is noted that the simplified diagram does not illustrate various connections, for example, power, ground, and interface connections to the various components; however, those skilled in the art will recognize the need for such connections and understand how to implement such connections, based on the components ultimately selected for use.

Referring to FIG. 1, the navigation system 10 includes a surgical aid component such as movable guide member 11, a power source or emitting device 17, and a control unit 16. The system may also include a surgical instrument 13 to be tracked with respect to the movable guide member 11. The movable guide member 11 and surgical instrument 13 can include sensor elements 12 and 14, respectively. The emitting device 17 emits a propagating signal to communicate with the sensors 12 and 14 to track the location of the surgical instrument 13 relative to the movable guide member 11. The emitting device 17 may also include a detection unit 17c to detect responses of the sensors 12, 14. Once the responses are detected by the detection unit 17c, the control unit 16 utilizes a multi-triangulation concept to calculate the position of the sensors 12 and 14 based on the detected responses to tracking signals emitted by the emitting device 17. The manner in which the emitting device 17 and/or detection unit 17c communicates with the sensors 12 and 14 to track the position thereof is well known in the art and is therefore only described generally. In some embodiments, it is possible that the functions of the emitter 17 and sensors 12 and 14 may be reversed and/or combined using sound engineering judgment to achieve the same or similar results. For example, it is possible for the sensors 12 and 14 to function as emitters rather than sensors, and it is possible for the emitter 17 to function as a sensor rather than an emitter. In either case, it is possible to utilize known triangulation methods to calculate and track the positions of the sensors 12 and 14 relative to the targeted surgical field using the configurations and techniques of the present general inventive concept. In other embodiments, the navigation system 10 may include an optional imaging device (not illustrated), such as an MRI unit, CT scanner, or other type of imaging device, to acquire pre-, intra-, or post-operative or real-time images of the patient 1, in order to determine location coordinates with respect to a fixed portion of the patient's body, for example, to obtain digital coordinates of the various components relative to the patient's maxilla or skull region 15.

Referring to FIG. 1, the emitting device 17 can generate a tracking signal which can be received by sensors 12 and/or 14. The tracking signal may take the form of an infrared light signal (IR), electromagnetic (EM) signal, Bluetooth signal, Wi-Fi signal, or other known or later developed wired or wireless signal. In the example embodiment of FIG. 1, it is presumed for convenience of description that the propagating signal is an LED light signal transmitted from the emitting device 17 to the sensors 12 and 14. In this embodiment, in order to track the location of the guide member 11 and/or surgical instrument 13, the sensors 12 and 14 can function as reflecting markers to transmit light signals received from the emitting device 17 to a detection unit 17c, such as a CCD camera device. Using the reflected LED signals, the detection unit 17c can determine the location of the sensors 12 and 14 based on characteristics such as intensity, refraction angle, etc. of the reflected LED signals, and can inform the control unit 16 of the location of the sensors in real time based on the characteristics of the reflected LED signals. In other embodiments, it is possible that the sensors 12 and 14 can include one or more emitting devices to emit LED signals directly from the sensors to the detection unit 17c. In this case, the position of the sensors 12, 14 can be directly tracked by the detection unit 17c by detecting and characterizing the LED signals emitted from the sensors directly, in which case the emitting device 17 may not be required. Those skilled in the art will appreciate that many other configurations and combinations of elements in addition to those illustrated in FIG. 1 could be used without departing from the broader scope of the present general inventive concept.

During typical dental or medical procedures, the patient's MRI or CT scans may be fed into the control unit 16 to compare the scanned MRI or CT images to anatomical landmarks or reference points fixed on the patient's head and face to calibrate a location of the fixed reference point relative to a target point for the procedure or surgery. In the embodiment of FIG. 1, the patient's maxilla 15 can be used as a fixed reference point. To register the fixed reference point, it is possible to calculate a position of the fixed reference point with respect to the targeted surgical field (e.g., mandible region) based on coordinates of the patient generated by the MRI or CT scans. It is also possible to directly register a location of the fixed reference point by mounting a fixed device, such as a screw device (not illustrated), adapted to include an integrated sensor device to correspond and define a fixed reference point of the patient's skull. The fixed sensor device can then be used to communicate with the emitting device 17 and/or detection unit 17c to calibrate the location of the fixed reference point relative to one or more other sensors or reference points of the patient. In this way, the fixed reference point 15 may be used as a positional reference frame to determine the relative position of the surgical instrument 13 with respect to the target point of the surgery, and to calibrate a position of the movable guide element 11.

To carry out a particular surgical process, it may be important to move the patient's mandible 19 during the process as indicated by the phantom lines and direction arrow illustrating movement of the mandible 19 as depicted in FIG.1. Here, the surgeon can attach a surgical aid component such as a movable guide member 11 adapted with a sensor array 12 to a portion of the patient's mandible to track movements of the patient's mandible 19, as illustrated in FIG. 1.

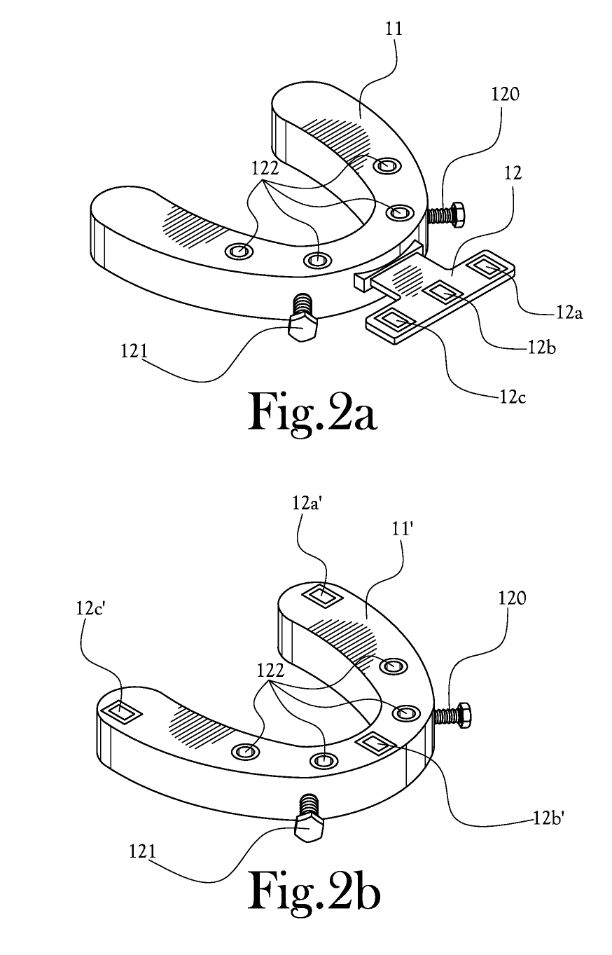

Referring to FIGS. 1 and 2A, the exemplary movable guide member 11 can be configured in the shape of a semicircular mouthpiece to fit precisely on the patient's mandible. The movable guide member 11 typically includes a series of holes 122 which the surgeon uses to locate and orient dental implants during oral surgery. The movable guide member 11 can be attached to the patient's mandible by way of fasteners 120 and 121. The fasteners 120, 121 may take the form of fixation screws, bolts, or pins, but the present general inventive concept is not limited thereto. Many other types of fastening devices or glues may be used to attach a guide member 11 and sensor 12 to these and/or other movable regions of the patient without departing from the broader scope of the present general inventive concept. For example, fixation methods such as intermaxillary fixation (IMF) methods, IMF screws, and the like, can be adapted to include a sensor device in accordance with the present general inventive concept to track movements of a movable region of the patient during a medical or dental procedure. It is possible to mount a sensor 12 to a guide member such as a bite plate device, secured to a lower jaw of the patient by screws. Moreover, although the example embodiment of FIG. 2A illustrates a mouthpiece-shaped guide member 11 to incorporate the sensor 12, the present general inventive concept is not limited to such configuration, and various other types of sensor arrangements may be used in connection with a variety of other types of fixation devices, methods, or splints to track and maintain a movable reference point during surgery. For example, it is possible to incorporate a sensor device into a locating pin or other fastening device, such as a surgical screw, and to attach the pin or screw to the targeted movable region of the patient to track the movable reference during a particular medical or dental (i.e., surgical) procedure. It is also possible to integrate RFID sensors, and/or other types of sensors, into a mesh-like bite plate device, where the sensors are disposed or integrated within the mesh construct of the device itself. The integrated device can then be attached to a movable region of interest, such as the patient's lower jaw, to track movements thereof during an operative procedure. The present general inventive concept is not limited to the exemplary configurations illustrated and described herein. To the contrary, a variety of other configurations and combinations of dental/medical devices can be adapted with a variety of different sensor technologies (e.g., swarming technology) to carry out the techniques of the present general inventive concept. For example, it is possible to utilize various combinations of sensor technologies, such as EM and/or optical, during a single operative procedure, depending on the particular components and instruments chosen and adapted for use.

Referring to the example embodiment of FIG. 2A, there is illustrated a perspective view of a typical movable guide member 11 adapted to include an array of sensor members 12a, 12b, and 12c to detect light emitted from the emitting device 17, in accordance with an example embodiment of the present general inventive concept. In this example embodiment, the sensors 12a, 12b, and 12c can function as reflecting markers to transmit light signals received from the emitting device 17 to a detection unit 17c. The detection unit 17c can continuously acquire the position of the sensors 12a, 12b, and 12c and can inform the control unit 16 of the location of the sensors in real time. The control system 16 can compute the position of the movable guide member 11 using a known multi-triangulation method based on information received from the sensors 12a, 12b, and 12c, and can display on display monitor 8 an image displaying the position of the movable guide member 11 with respect to various other components, structures, and reference points of the navigation system 10.

Referring to FIGS. 1 and 2A, the sensors 12a, 12b, and 12c can be configured to extend from an outer surface of the guide member 11 to help maintain consistent line-of-sight between the sensors 12a, 12b, 12c and the light emitting device 17. Although FIGS. 1 and 2A depict an oral surgery configuration, those skilled in the art will appreciate that the present general inventive concept is not limited to the embodiments of FIGS. 1 and 2A, and that many other shapes and sizes of guide members 11 and sensors 12a, 12b, 12c may be used to facilitate mounting of such devices on other parts of the body, internally and externally, and may be used in connection with other types of surgeries where it is useful to maintain a movable reference to help locate surgical instruments when the target anatomical structure is moved during surgery.

In the case of dental implants, for example, it is possible to mount a sensor array 12 to the movable guide member 11 to facilitate tracking of the guide member 11 as the mandible is moved, enabling the surgeon to maintain consistent and proper positioning of the surgical instrument 13 with respect to the mandible even when the mandible is moved during surgery.

In the embodiment of FIG. 1, the surgeon attaches the movable guide member 11 and sensor 12 to the target point, such as the patient's mandible 19 as illustrated in FIG. 1. During a surgical procedure, the control unit 16 can track the location of the movable guide member 11 and the surgical instrument 13 in real time, enabling the surgeon to maintain proper positioning of the surgical instrument 13 with respect to the target point even when the movable guide member 11 is moved during surgery.

During a surgical procedure, the surgeon may move the surgical instrument 13 with respect to the targeted surgical region of the patient, for example the mandible 19 area as illustrated in FIG. 1. As the surgeon is moving the surgical instrument 13, the control unit 16 can track the location of the surgical instrument 13 via the sensors 14 mounted on the surgical instrument 13. The control system 16 can interpret the response signals of the sensor 14 to compute the position of the surgical instrument 13 using a known multi-triangulation method based on response signals of the sensors 14, and can display on display monitor 8 an image displaying the position of the surgical instrument 13 with respect to the targeted region of the patient. These techniques enable a surgeon to track the relative positions of the movable guide member 11 and surgical instrument 13 in the targeted surgical field, even when the movable guide member 11 is moved during the surgical process.

Referring to FIG. 1, in the case where the emitting device 17 emits infrared light signals, it is important that the sensors 12 and 14 remain in the visual field of the emitted light signals to help produce consistent and accurate locations of the movable guide member 11 and surgical instrument 13 in the control unit 16 as the surgical instrument 13 and guide member 11 are moved during surgery. However, in cases where the emitting device does not emit light signals but instead emits EM or other types of RF or wireless signals, it is not as important to maintain the sensors 12 and 14 in the visual line-of-sight of the emitted signals, as EM and other types of RF signals have the ability to penetrate and communicate with sensors that are not directly in the visual line-of-sight of the EM or RF source.

FIG. 2B is a perspective view of guide member including sensor members in accordance with another example embodiment of the present general inventive concept, for example, in a case where the emitting device 17 emits EM or other RF-based signals.

Referring to FIG. 2B, in a case where the emitting device 17 emits EM or other RF-based signals, the sensors of the movable guide member 11′ can include an array of detectors, such as radio frequency identification (RFID) sensors 12a′, 12b′, and 12c′, to communicate with the EM signals emitted from the emitting device 17. Unlike the configuration of FIG. 2A, the RFID sensors 12a′, 12b′, and 12c′ can be mounted internally with respect to the guide member 11′ as illustrated in FIG. 2B. The RFID sensors can be mounted within the internal structure of the guide member 11′ since it is not as important to maintain a direct line-of-sight between the sensors and the emitting device 17 due to the penetrating characteristics of EM and other types of RF signals. In operation, the RFID sensors 12a′, 12b′, and 12c′ function to interact with the electromagnetic field generated by the emitting device 17, and the control unit 16 can recognize any disruptions in the magnetic field caused by the RFID sensors, enabling the system's computer, which has special tracking software, to recognize the location of the RFID sensors and its location in the surgical field using a known multi-triangulation concept based on the interaction of the RFID sensors 12a′, 12b′, and 12c′ with the electromagnetic field. Similar to the embodiment of FIG. 2A, the control unit 16 can compute the position of the movable guide member 11′ in real time based on this information, and can display on display monitor 8 an image displaying the position of the movable guide member 11′ with respect to various other components, structures, and reference points of the navigation system 10.

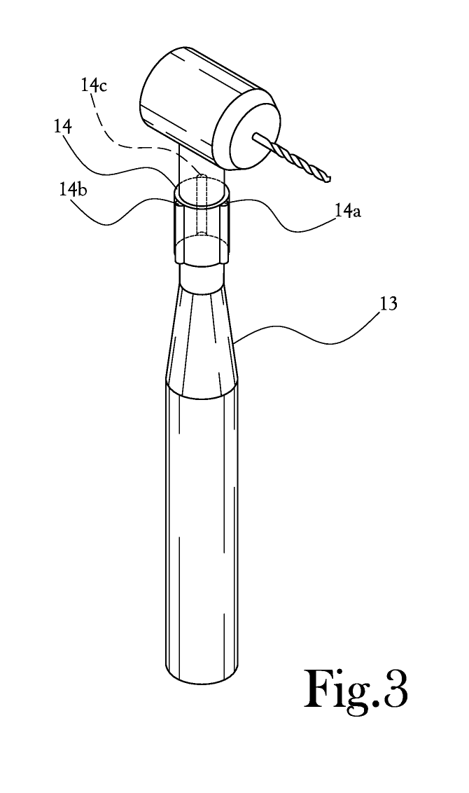

FIG. 3 is a perspective view of an exemplary surgical instrument 13 including a sensor array 14 configured in accordance with an example embodiment of the present general inventive concept.

Referring to FIG. 3, the surgical instrument 13 includes a sensor array 14 including sensors 14a, 14b, and 14c. These sensors are configured to respond to propagating signals emitted from the emitting device 17 to track the location of the surgical instrument in the surgical field, in the manners discussed above. As with sensors 12a, 12b, and 12c, sensors 14a, 14b, and 14c can be configured to interact with LED, EM, Wireless, WiFi, Bluetooth, IR, and/or other types and combinations of wired or wireless signals in known ways to track the location of various components associated with the sensors.

To facilitate attachment of the sensor array 14 to the surgical instrument, the sensor array may be mounted in the form of a ring-like shape to fit around a shaft or neck region of the surgical instrument 13, as illustrated in FIG. 3. Such a configuration is easily adaptable to any number of different shaped and sized surgical instruments. However, those skilled in the art will appreciate that the specific means of mounting the sensors to the various components can be chosen with sound engineering judgment, and a variety of mounting shapes and configurations could be used without departing from the broader scope of the present general inventive concept. For example, the sensors 14a, 14b, and 14c could be integrally mounted and formed in the surgical instrument 13 as a single body to communicate with the propagating signal without sacrificing proper positioning of the surgical instrument 13 with respect to the surgical field. Using the responses of the sensors 14a, 14b, and 14c, the control unit 16 can calculate the position of the surgical instrument 13 relative to the movable reference region and can track and compare the relative movements of the guide member 11 with respect to the surgical instrument 13. It is possible to include a slot or other type of holding means in one or more of the exemplary devices of the navigation system to hold a microSD card or other memory device to store or upload data to/from the navigation system.

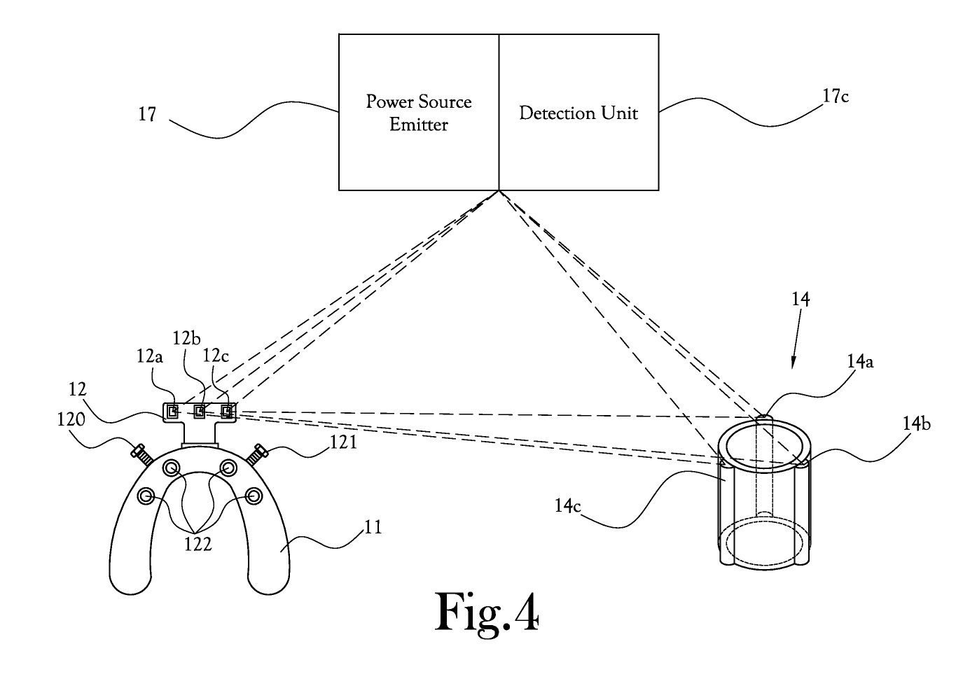

Referring to FIG. 4, it is possible to configure the sensors 12 and 14 to communicate with each other, in addition to communicating with the emitter device 17 and/or detection unit 17c, to provide additional information about the relative positions of the respective guide member 11 and surgical instrument 13. In this regard, the sensors 12 and 14 are not required to be the same or similar types of devices, but instead may be different, wherein the sensors independently interact with one or more of the emitting devices 17 and/or detection unit 17c to track location information of the respective sensors. For example, one of the sensors 12 could be configured to include an EM source and a light reflector sensor, and the other sensor 14 could be configured to include an RFID receptor to interact with the EM field generated by sensor 12. In such a case, the emitter device 17 and detection unit 17c could be adapted to track the location of sensor 12 by characterizing the light reflected by sensor 12, and the control unit 16 could be adapted track the relative distance between the sensors 12 and 14 by detecting disruptions in the EM field caused by movement of the RFID receptor of sensor 14. A variety of other types and combinations of sensors could also be used.

FIG. 4 is a simple diagram illustrating a light source and light detector in communication with sensor arrays 12, 14 in accordance with an example embodiment of the present general inventive concept. In this embodiment, a minimum of three points of reference are used, corresponding to three sensors on each device (12a, 12b, 12c and 14a, 14b, 14c). Typically, the sensors 12a, 12b, 12c and 14a, 14b, 14c can communicate with the power source 17 and/or detection unit 17c to provide information regarding the location of the respective devices, as indicated by the dotted lines extending between the sensors and the power source 17 and detection unit 17c. It is also possible that the sensors 12a, 12b, 12c can communicate directly with the other sensors 14a, 14b, 14c to provide information about the relative positions of the devices, as indicated by the dotted lines extending between the sensor arrays 12 and 14. For example, the sensors 12a, 12b, and 12c could be configured to include an EM source to emit a tracking signal to the sensors 14a, 14b, and 14c, and the sensors 14a, 14b, and 14c could be configured to include an RFID receptor configured to interact with the EM field generated by the EM source based on the position of the RFID receptors. Accordingly, disruptions or changes to the EM field caused by movement of the RFID receptors can be detected by the detection unit 17c and fed to the control unit 16 (FIG. 1) to calculate and display location information about the relative positions of the sensors. Moreover, the use of RFID, Bluetooth, IR, EM, LED, or other types of sensors can be interchanged, mixed, or combined for use with different devices and applications, without departing from the broader principles and scope of the present general inventive concept. For example, swarming technology can be used to implement a variety of different sensor technologies (e.g., EM and/or optical) on a variety of different surgical components and regions of interest to track movements thereof during single or multiple operative procedures of a patient.

It is also possible to utilize thermography in conjunction with the navigation techniques of the present general inventive concept to identify other structures in and around the surgical region of interest such as nerves, arteries, veins, and the like. For example, after the RFID sensors track and identify the location of teeth or other structures in a surgical region of interest, such as the mandible, it is possible to identify the location of nerves, arteries, or veins in the mandible using thermography, thus providing additional navigational information to supplement the information provided from the multi-triangulation techniques of the present general inventive concept. In other words, it is possible to incorporate thermal imaging cameras into, or in combination with, the exemplary sensors of the present general inventive concept in order to detect variations in the infrared radiation of various body parts and to display thermographic images thereof. In this way, if the surgeon knows that the artery, vein, or nerve runs along with the vein, the use of thermography can be used to identify where the canal is, thus providing additional location information in addition to the information provided by the RFID or other sensors. Accordingly, not only can the multi-triangulation concepts of the present general inventive concept be used to indicate where a boney indentation is in the bone, but thermography concepts can also be incorporated into the navigation system of the present general inventive concept to help identify and locate the nerve, artery, and/or vein during surgery.

While the present general inventive concept has been illustrated by description of example embodiments and while the illustrative embodiments have been described by referring to the drawings, it is not the intention of the applicant to restrict or in any way limit the scope of the appended claims to the illustrative examples. Additional advantages and modifications of the present general inventive concept will readily appear to those skilled in the art. The present general inventive concept in its broader aspects is therefore not limited to the specific details, representative apparatus and methods, and illustrative examples illustrated and described. Accordingly, departures may be made from such details without departing from the spirit or scope of applicant's general inventive concept.

What is claimed is:

1. A navigation system to track positions of surgical instruments during surgery of a patient, comprising:
a power source to emit a tracking signal during surgery of a patient;
a first sensor mounted to a movable region of the patient to respond to the emitted tracking signal; and
a control unit to track a position of the movable region relative to a fixed region of the patient as the movable region moves with respect to the fixed region, based on the response of the first sensor.

2. The navigation system of claim 1, further comprising:
a second sensor mounted to a surgical instrument to respond to the emitted tracking signal such that the control unit tracks a position of the surgical instrument relative to the movable region as the surgical instrument and movable region move with respect to the fixed region, based on the responses of the first and second sensors.

3. The navigation system of claim 2, wherein the first and second sensors each comprise at least three RFID receptors to interact with the emitted tracking signal, and the control unit tracks the position of the surgical instrument relative to the movable region using a triangulation calculation based on the interaction of the at least three RFID receptors.

4. The navigation system of claim 2, further comprising:
a detection unit to detect the responses of the first and second sensors such that the control unit tracks the movement of the movable region and the surgical instrument based on the detected responses.

5. The navigation system of claim 4, wherein the first and second sensors each comprise at least three LED reflectors to reflect the emitted tracking signal, and the control unit tracks the position of the surgical instrument relative to the movable region using a triangulation calculation based on the reflected signals of the at least three LED reflectors.

6. The navigation system of claim 2, wherein the first sensor comprises an emitting unit to emit a second tracking signal to the second sensor, and the second sensor comprises a receptor unit to respond to the second tracking signal such that the control unit tracks the movement of the surgical instrument relative to the movable region based on the response of the receptor unit to the second tracking signal.

7. The navigation system of claim 1, wherein the first sensor comprises at least three RFID, Bluetooth, LED, or WiFi receptors to interact with the emitted tracking signal, and the control unit tracks the position of the movable region using a triangulation calculation based on the interaction of the at least three receptors.

8. The navigation system of claim 1, further comprising:
a surgical aid component fixedly mounted to the movable region, wherein the first sensor is coupled to an outer surface of the surgical component and is oriented to maintain a visible line of sight with the emitted tracking signal.

9. A navigation system to track positions of surgical instruments during surgery of a patient, comprising:
a detection unit to detect an LED signal;
a first sensor mounted to a movable region of the patient to emit a first LED signal to be detected by the detection unit; and
a control unit to track a position of the movable region relative to a fixed region of the patient as the movable region moves with respect to the fixed region, based on the detected first LED signal.

10. The navigation system of claim 9, further comprising:
a second sensor mounted to a surgical instrument to emit a second LED signal to be detected by the detection unit such that the control unit tracks a position of the surgical instrument relative to the movable region as the surgical instrument and movable region move with respect to the fixed region, based on the detected first and second LED signals.

11. The navigation system of claim 10, wherein the first and second sensors each comprise at least three LED emitters to respectively emit first, second, and third light signals to be detected by the detection unit, such that the control unit tracks the position of the surgical instrument relative to the movable region using a triangulation calculation based on the detected first, second, and third light signals.

12. The navigation system of claim 10, wherein the first sensor comprises an emitting unit to emit a tracking signal to the second sensor, and the second sensor comprises a receptor unit to respond to the tracking signal such that the control unit tracks the movement of the surgical instrument relative to the movable region based on the response of the receptor unit to the tracking signal.

13. The navigation system of claim 9, further comprising:
a surgical component fixedly mounted to the movable region, wherein the first sensor is coupled to an outer surface of the surgical component to maintain a visible line of sight with the light detector as the movable region is moved during the surgery.

14. A method of tracking positions of surgical instruments during a surgical process of a patient, comprising:
emitting tracking signals to a targeted region of the surgical process;
coupling a first sensor to a movable region of the patient such that the first sensor responds to the emitted tracking signals; and
tracking a position of the movable region relative to a fixed region of the patient as the movable region moves with respect to the fixed region, based on the response of the first sensor.

15. The method of claim 14, wherein a location of the fixed region is based on a scanned image of the patient.

16. The method of claim 14, further comprising:
coupling a second sensor to a surgical instrument to be used in the surgery such that the second sensor responds to the emitted signal;
tracking a position of the surgical instrument relative to the movable region as the surgical instrument and movable region move with respect to the fixed region, based on the responses of the first and second sensors; and
displaying an image of the relative positions of the surgical instrument and movable region.

17. The method of claim 16, wherein the first sensor comprises an emitting unit to emit a second tracking signal to the second sensor, and the second sensor comprises a receptor unit to respond to the second tracking signal such that the control unit tracks the movement of the surgical instrument relative to the movable region based on the response of the receptor unit to the second tracking signal.

18. The method of claim 14, wherein the coupling of the first sensor to the movable region of the patient comprises:
fixedly mounting a surgical aid component to the movable region; and
coupling the first sensor to the surgical aid component.

19. The method of claim 18, wherein the first sensor is coupled to an outer surface of the surgical aid component and is oriented to maintain a visible line of sight with the emitted signals as the movable region moves with respect to the fixed region during the surgical process.

20. The method of claim 16, wherein the first sensor comprises at least three RFID, Bluetooth, LED, or WiFi receptors to interact with the emitted tracking signals, and the control unit tracks the position of the movable region using a triangulation calculation based on the interaction of the at least three receptors.
